(19) United States
(12) Plant Patent Application Publication
(10) Pub. No.: US 2018/0160600 P1
(43) Pub. Date: Jun. 7, 2018

(54) Baptisia plant named 'Lavender Stardust'

(50) ;

(71) Applicant: Hans A. Hansen, Zeeland, MI (US)

(72) Inventor: Hans A. Hansen, Zeeland, MI (US)

(73) Assignee: Walters Gardens, Inc., Zeeland, MI (US)

(21) Appl. No.: 15/530,089

(22) Filed: Dec. 1, 2016

(51) Int. Cl. A01H 5/02

(52) U.S. Cl. PLT/263.1

(57) ABSTRACT

A new and distinct plant cultivar of hardy herbaceous false indigo named Baptisia ‘Lavender Stardust’ characterized by lilac purple flowers on long spikes quickly becoming dusty lavender purple with pale yellow keel. The new plant has a broad, mounded, multi-stemmed, winter-hardy habit with glaucous medium-green tri-foliate foliage and is suitable for landscaping as a specimen or en masse.

Botanical classification: Baptisia hybrid.

Cultivar designation: ‘Lavender Stardust’.

### BACKGROUND OF THE INVENTION

The present invention relates to a new and distinct cultivar of hybrid Baptisia plant, botanically known as Baptisia ‘Lavender Stardust’ and will be referred to hereafter by its cultivar name, ‘Lavender Stardust’. The new cultivar represents a new false indigo, a hardy herbaceous perennial grown for landscape and cut flower use.

The new invention arose from an ongoing breeding program of the inventor at a nursery in Waseca, Minn. with continued evaluation at a wholesale perennial nursery in Zeeland, Mich. with the specific intention of improving garden worthiness of perennial false indigo plants with a wider variety of flower colors and improved garden habit.

Baptisia ‘Lavender Stardust’ was a single seedling selection from a cross between a proprietary unreleased selection of Baptisia albescens (not patented) as the female or seed parent times a proprietary unreleased hybrid of Baptisia minor times Baptisia australis as the male or pollen parent.

Seeds were collected from the individual selected female plant in fall of 2004 at the isolation block in Waseca, Minn., USA by the inventor. The seeds were sown by the inventor in Waseca, Minn. in the fall of 2004 and the initial selection made in the spring of 2008 at the same nursery in Waseca.

No plants of Baptisia ‘Lavender Stardust’ have been sold, under this or any name, in this country or anywhere in the world, prior to the filing of this application, nor has any disclosure of the new plant been made prior to the filing of this application with the exception of that which may have been disclosed within one year of the filing of this application and was either derived directly or indirectly from the inventor.

‘Lavender Stardust’ was initially asexually propagated by stem cuttings at a nursery in Waseca, Minn., USA in 2008. The resultant plants have demonstrated that the new plant has remained stable and true to type in successive generations of asexual propagation.

The nearest comparison plant known to the inventor is ‘Blueberry Sundae’ U.S. Plant Pat. No. 23,891. Compared to ‘Blueberry Sundae’ the new plant is taller and broader in habit, and the flowers of the new plant are lighter lavender-purple on darker green stems. Compared with typical Baptisia australis the flowers on the new plant have a more lavender hue. Compared with Baptisia minor the new plant is taller in habit, and compared with Baptisia albescens the new plant has denser fuller habit and lavender purple flower compared to the creamy white of Baptisia albescens.

The new plant differs from all Baptisia known to the inventor in the following combined traits:
1. Lilac purple flowers on long spikes.
2. Flowers lightening quickly to dusty lavender purple with a pale yellow keel.
3. Broad, mounded, multi-stemmed, winter-hardy habit.
4. Glaucous, medium-green, tri-foliate foliage.

### BREF DESCRIPTION OF THE DRAWINGS

The photographs of the new plant demonstrate the unique traits of the new plant and the overall appearance. The colors are as accurate as reasonably possible with color reproductions. Variation in ambient light spectrum, source and direction may cause the appearance of minor variation in color. The accompanying photograph is of a ten-years-old plant growing in an open full-sun trial garden in Zeeland, Mich.

FIG. 1 shows a close-up of the flower scape.

FIG. 2 shows the habit of a plant in full flower.

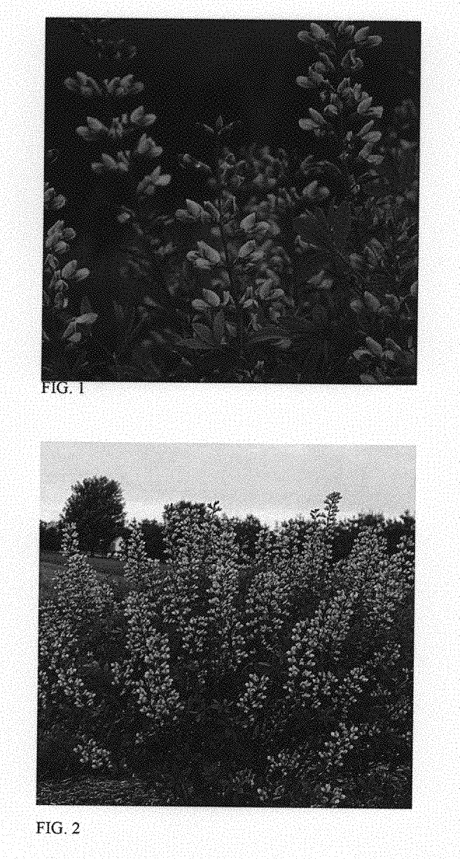

### DETAILED BOTANICAL DESCRIPTION OF THE PLANT

The following is a detailed description of ten-year-old plants of the new cultivar as grown outdoors in a trial plot at a wholesale nursery in Zeeland, Mich. Plants of the new cultivar have not been tested under all possible conditions. The phenotype may vary with changes in environment, climate, and cultural conditions without change however in the genotype. The color references are in accordance with the 2001 edition of The Royal Horticultural Society Colour Chart except where general color dictionary terms are used.
Plant habit: perennial, compact, well-branched, many-stemmed, vase-shaped growth habit with long inflorescences held above the foliage;
Plant size: stems and crown about 35 cm across at soil level, about 105 cm tall from soil to top of flowers and 170 cm wide just below initial flowers;
Roots: fibrous, well-branched, long, deeply rooted;
Root color: nearest RHS 161D;
Propagation method: stem cuttings, rooting in about three weeks;
Growth rate: moderate, similar to other Baptisia;
Stems: rigid and upright to outwardly; highly glaucous; glabrous; cylindrical with longitudinal furrows; lower two to three nodes without leaves or branches; normally 22 to 28 stems per plant; main stem to about 12.0 mm diameter at base and 105 cm tall to top of inflorescence, average about 95 cm long and about 10.0 mm diameter; normally about four alternately-arranged primary branches at 55 to 65 degree angle above horizontal, up to 25.0 cm long and 4.0 mm diameter, average for primary branches about 18.0 cm long and 3.5 mm diameter, smaller distally; and three alternately-arranged secondary branches per stem of about 12.0 cm long and 3.0 mm diameter, averaging about 10.0 cm long and 3.0 mm diameter;
Stem color: base nearest RHS N138A;
Stem scales: at stem nodes; slightly glaucous; dehiscing to leave behind thin scar; about 4.2 cm long and 17 mm wide at base and 22 mm wide in center; frequently with apical three leaflets about 4.0 mm long and 2.0 mm wide in center of retuse apex with sharply pointed sides; truncate base;
Stem scale color: between RHS 138C and RHS 138B before dehiscing and nearest RHS N200A after dehiscing;
Internodes: up to 10.0 cm apart between lowest branches, average about 7.0 cm;
Foliage: alternate on stem; ternate to palmately compound with three leaflets; outer two leaflets at about 65 to 80 degree angle to middle leaflet, increasing with leaf age; up to 5.0 cm long and 10.0 cm wide, average 4.0 cm long and 6.5 cm wide;
Leaflet: three, oblanceolate; apex acute, base attenuate; margins entire; petiolate; adaxial surface matte, scaberulosc abaxial; size up to 5.0 cm long and 2.1 cm wide, average about 3.8 cm long and 1.8 cm wide;
Leaflet color: newly expanding blend between RHS N144C and RHS 144B on upper and lower surfaces; mature upper and lower surface between RHS 139B and RHS 139A;
Venation: pinnate, glabrous, thin, not conspicuous;
Vein color: newly expanding foliage midrib nearest RHS N144D above and nearest RHS 145D below; main c.cntci vein nearest RHS 139D above and lighter than RHS 142D or RHS 145D below; secondary veins same color as surrounding leaf tissue;
Petioles: glabrous; slightly glaucous; concaved facing upward; up to about 1.1 cm long and 2.0 mm wide average 1.0 cm long and 1.5 mm wide;
Petiole color: nearest 138A on upper and lower surfaces;
Stipules: narrowwly lanceolate, acute apex with base truncate to stem; up to 2.4 cm long and 11.0 mm wide, average 2.0 cm long and 10.0 mm wide with largest stipules below primary branches and decreasing distally and on secondary branches;
Stipule color: nearest RHS 138A both surfaces;
Flower: zygomorphic, papilionaceous, non-secund, held at about 30 degree angle above horizontal; about 45 flowers per main raceme and about 15 per secondary branch; seasonally effective for about 3 to 4 weeks beginning in late May to early June in Zeeland, Mich.; individual flower remain effective and on raceme for about four days; individually about 2.3 cm long, 1.8 cm tall and 1.4 cm wide at tallest and widest portions; an upper banner, a lower keel made up of two lobes folded around gynoecium and androecium; and two lateral wings or alae laterally appressed against keel;
Flower fragrance: none detected;
Peduncle: rounded with vertical ridges and furrows; glaucous; glabrous; from first flower to apex about 42 cm long; diameter at the base of first flower about 5.0 mm and about 2 mm diameter at the apex;
Peduncle color: nearest RHS N138A at base and nearest RHS N138A with heavy tinting of between RHS N187B and RHS N187A in upper portions, where exposed to higher light levels;
Pedicel: round in cross section, glabrous, slightly glaucous; about 15.0 mm long and 1.0 mm diameter;
Pedicel color: nearest RHS N138B with tinting of nearest RHS N187B in upper portions or where exposed to more light;
Calyx: campanulate, five-lobed with top lobe only dissected about 1.0 mm and other lobes dissected about 3.0 mm deep, or four lobed with top lobe completely fused; compressed along petals; apex obtuse, base fused in basal two thirds; margins entire; slightly glaucous; about 9.0 mm long and 5.0 mm diameter; persists after petal drop;
Calyx color: abaxial nearest RHS N138A with glaucous coating giving it a color of between RHS 122 B and RHS N138C; adaxial nearest RHS 144A;
Buds one day prior to anthesis: oblong elliptic, flatted vertically; about 2.0 cm long and 11.0 mm tall and 6.0 mm wide;
Bud color: about two days prior to opening exposed banner petal nearest RHS 93C, exposed keel petal nearest RHS 91C and alae petals enclosed in banner petal;
Petals: five; with a lower fused keel, an upper banner, and two lateral wings or alae; keel comprised of two sections that are folded around stamens and pistil;
   Banner petal.—conduplicate, curved upward and backward and pinched in the middle; apex retuse, base claw-like, margin entire; about 1.8 cm long, 1.6 cm across at widest and 7.5 mm tall; banner dorsal side color lighter than RHS 91D at perimeter margin with the inner center portion nearest RHS 91A; young ventral side apex nearest RHS 91B lightening to between RHS 91C and RHS 91D, base claw nearest RHS 145A;
   Keel.—comprised of two main lobes that are folded around stamens and pistil; fused in the distal one-third with the apex emarginate or retuse and the bases separate and claw-like; margin entire; top edge about one-third of the way from base has 2.0 mm smaller lobe pointing toward base; about 20 mm long 0.5 cm across and 9.0 mm tall with claw base narrowed to 1.0 mm wide for the proximal 5.0 mm; color of outer keel sides between RHS 91C and lighter than RHS 91D with apex lighter than RHS 91D; inner color same as outer color;
   Alae.—papilionaceous corolla appendage with rounded apex and claw-like base; with 2.0 nun lobe pointing toward base and about one-third of the way from base; about 21 mm long and 8.0 mm tall with the claw narrowed to 2.0 mm deep for the distal 5.0 mm; alae outside color nearest RHS 11D; color of lobe nearest RHS 11D on inside and outside, claw color nearest RHS 145C on inside and outside;
Receptacle: disk-shaped, about 3.3 mm diameter and 1.2 mm depth; color nearest RHS 138A;
Gynoecium: one, with superior ovary,
   Pistil.—about 22.0 mm long and 1.5 mm wide;
   Style.—curved at tip, about 8.0 mm long and less than 1.0 mm diameter; color nearest RHS 145C;
   Stigma.—less than 0.5 mm diameter; color nearest RHS 145D;
   Ovary.—superior suspended by stipe; about 8 mm long and about 1.5 mm in diameter; color nearest RHS 144A;
   Stipe.—about 7.0 mm long and 1.0 mm diameter; color nearest RHS 144B:
Androecium:
   Stamens.—ten, not united, about 20.0 mm long and 1.0 mm diameter;
   Filament.—1.9 cm in length and less than 1 mm in diameter; slightly curved upward at tip; filament color between RHS 145D toward base and RHS 145C at distal region;
   Anther.—dorsifixed, oblong; about 2.0 mm long and 0.8 mm wide; color nearest RHS 15A;
   Pollen.—spherical; abundant; color nearest RHS 15A;
Fruit: bivalve inflated pod; glabrous; about 3.0 cm long, 1.2 cm across and 0.8 cm thick; with thin linear terminal beak about 5.0 mm long and about 0.3 mm thick;
Fruit color: at maturity nearest N200A;
Seeds: about 11 per flower (open pollinated); reniform; glabrous; about 4.0 mm long, 2.5 mm across and 1.5 mm thick; color between RHS 165A and RHS 165B;
Hardiness: to USDA zones 4 to 9; tolerant of heavy clay or light loamy-sand soils; able to withstand drought conditions once established;
Diseases: susceptibility or resistance to diseases beyond that typically found in other false indigo plants has not been observed;

It is claimed:

1 A new and distinct plant cultivar of hardy herbaceous false indigo named Baptisia ‘Lavender Stardust’ as herein described and illustrated.
